(19) United States
(12) Patent Application Publication
(10) Pub. No.: US 2004/0029198 A1
(43) Pub. Date: Feb. 12, 2004

(54) Method of analyzing ataxia-telangiectasia protein

(76) Inventors: Richard A. Gatti, Sherman Oaks, CA (US); Anthony W. Butch, Pacific Palisades, CA (US); Helen H. Chun, Woodland Hills, CA (US); Xia Sun, Beijing (CN)

Correspondence Address: KNOBBE MARTENS OLSON & BEAR LLP, 2040 MAIN STREET, FOURTEENTH FLOOR, IRVINE, CA, 92614, US

(21) Appl. No.: 10/431,632

(22) Filed: May 5, 2003

Related U.S. Application Data
(60) Provisional application No. 60/379,841, filed on May 9, 2002.

(51) Int. Cl. G01N 33/574; G01N 33/53; G01N 33/537; G01N 33/543

(52) U.S. Cl. 435/7.23; 435/7.92

(57) ABSTRACT

The present disclosure concerns methods for recombinantly producing functional ataxia-telangiectasia (ATM) protein, methods for isolating recombinant functional ATM protein, and uses of ATM protein including diagnosing a patient for A-T and/or susceptibility to various conditions including cancer, particularly breast cancer, neurological disorders, and heart disease. Specifically, a method is disclosed for determining the amount of ATM protein in a patient's cells, comparing that amount to a reference, and forming a diagnosis.

### RELATED APPLICATIONS

This application claims priority from U.S. Provisional Application No. 60/379,841 entitled METHOD OF ANALYZING ATAXIA-TELANGIECTASIA PROTEIN filed on May 9, 2002. The subject matter of the aforementioned application is hereby incorporated by reference in its entirety.

### GOVERNMENTAL SUPPORT

This invention was made with Government support by Grant No. NS35322, awarded by the National Institutes of Health. The Government may have certain rights in this invention.

### FIELD OF THE INVENTION

The present invention relates to diagnosing ataxia-telangiectasia and/or cancer susceptibility in patients using an Ataxia-telangiectasia protein. In particular, the disclosure describes construction of a recombinant vaccinia virus expressing functional ATM, purification of the protein from infected HeLa cells, demonstration of activity of the purified protein by means of in vivo and in vitro assays, and the diagnosis of patients for ataxia and/or cancer susceptibility.

### BACKGROUND OF THE INVENTION

Ataxia-telangiectasia (A-T) is a genetic recessive disorder that affects 1 in 40,000 to 100,000 births. Patients are affected by a large range of symptoms including telangiectasia (dilation of blood vessels) on the eyes, face, and shoulders, ataxia (loss of balance), neurodegeneration, cerebellar degeneration, ocular telangiectasia, radiosensitivity, cancer predisposition, immunodeficiency, and premature aging. A-T cells display cell cycle checkpoint defects, chromosomal instability, and sensitivity to ionizing radiation. A-T results only in individuals who are homozygous for the A-T gene mutation, but carriers of A-T (individuals who are heterozygous for the A-T gene mutation) often exhibit adverse health effects as well. In particular, carriers of A-T have increased susceptibility to various cancers, particularly breast cancer, as well as heart disease, compared to their homozygous normal counterparts. In studying the relationship between A-T and breast cancer, Waha et al. analyzed ATM transcripts and found low concentrations in breast carcinomas, intermediate levels in benign lesions and high levels in normal breast tissue, concluding that the ATM gene may contribute to the development and/or malignant progression of breast carcinomas (Waha et al. (1998) Int J Cancer 78(3):306-9). Djuzenova et al. examined cells from healthy donors, breast cancer patients, A-T heterozygotes and A-T homozygotes and concluded that the cells of individuals from both A-T groups exhibited increased sensitivity to DNA damage induced by x-irradiation (Djuzenova et al. (1999) Lab Invest 79(6):699-705). In a statistical study of patients, Broeks et al. reported a nine-fold increase in breast cancer risk among A-T heterozygotes (Broeks et al. (2000) Am J Hum Genet 66(2):494-500). More recently, Geoffroy-Perez et. al. reported a 3.6-fold increase in breast cancer risk among A-T heterozygotes (Geoffroy-Perez et. al. (2002) Int J Cancer 99(4):619-623). Numerous other investigations have examined the connection between A-T and breast cancer. See, e.g., Yuille et al. (1998) Recent Results Cancer Res 154:156-73; Meyn (1999) Clin Genet 55(5):289-304; Khanna (2000) J Natl Cancer Inst 92(10):795-802; Geoffroy-Perez et al. (2001) Int J Cancer 93(2):288-93. Some research also indicates an increased susceptibility to ischemic heart disease for A-T heterozygotes. See e.g., Su et al. (2000) Ann Intern Med 133(10):770-8; Swift et al. (1991) N Engl J Med 325(26):1831-6. It is estimated that approximately 0.5% to 1% of the general population are carriers of A-T.

The A-T gene, cloned by positional cloning (Savitsky et al (1995) Hum. Mol. Genet. 4: 2025-2032) encodes a 370 kDa protein kinase known as “ataxia-telangiectasia, mutated” (ATM) involved with the DNA double-stranded break response mechanism and initiation of repair, which are events responsible for maintaining the genomic integrity of the cell. Activation of ATM has effects on multiple signal transduction pathways related to cell cycle checkpoints and DNA damage repair. Complete genomic sequence (184 kb) of the A-T gene, also known as the ATM gene, is disclosed at GenBank Accession No. U82828 (Platzer et al. (1997) Genome Res. 7 (6), 592-605). ATM mRNA is disclosed at GenBank Accession No. U33841 (Savitsky et al (1995) Hum. Mol. Genet. 4: 2025-2032). Cloning, sequences, and organization of the A-T gene are disclosed, inter alia, in U.S. Pat. Nos. 6,265,158, 6,211,336 and 5,858,661 to Shiloh et al., and mutations in the A-T gene are disclosed in U.S. Pat. No. 5,955,279 to Gatti et al.

ATM is a serine/threonine kinase that targets many substrates including p53, RPA, MDM2, NBS1, Chk2, RPA, BRCA1, and other substrates that are postulated but currently unknown. (Gatti et al, (2001) in Metabolic and Molecular Bases of Inherited Disease, 8^{th} Ed, Scriver et al. Eds, pp 705-732) ATM is a member of a family of large kinases containing a C-terminal end homologous to the phosphatidylinositol 3-kinase domain. These proteins play a role in cell cycle checkpoint or DNA damage repair. Other proteins in this family include Rad 3, Mec1p, Mei-41, Rad 50, Tell and DNA-PK.

Many aspects of ATM function have been elucidated, but little is known about the structure due to difficulties in isolating ATM. Only a few domains have been identified based on protein homology (Savitsky, K., et al. (1995) Human Molecular Genetics 4: 2025-2032) and biochemical activity (Shafman, T., et al. (1997) Nature 386: 520-523; Banin, S., et al. (1998) Science 281:1674-1677; Canman, C., et al. (1998) Science 281: 1677-1679).

Over-expression of ATM has been difficult to accomplish due to the instability of the cDNA and the large protein size. Baculovirus expression and protein purification has been attempted (Scott et al. (1998) Biochem Biophys Res Comm 245:144-148; Ziv, et al. (1997) Oncogene 15: 159-167), but a high protein yield was difficult to obtain. When ATM was over-expressed in insect cells, only a fraction of recombinant protein was found in the soluble portions of cell preparations, and the majority of the protein was associated with cellular membranes (Ziv et al. (1997) Oncogene 15, 159-167). In 100 ml of infected insect cells, only 20 ng of ATM was produced (Scott et al. (1998) Biochem Biophys Res Comm 245: 144-148), whereas expression of other recombinant proteins often results in recovery of milligram amounts of protein.

A DNA requirement in ATM activation has been reported, but has been disputed. Banin et al. and Canman et al. reported ATM kinase activity against p53 substrate, where the activity was independent of DNA. (Banin et al. (1998) Science 281: 1674-1677; Canman et al. (1998) Science 281: 1677-1679) Chan et al. determined that ATM activity was manganese-dependent and DNA-independent, except when ATM was phosphorylating RPA, in which case DNA was required. (Chan et al (2000) Jnl Biol Chem 275: 7803-7810) Smith et al. used DNA-iron oxide particles as their final purification step to isolate ATM from HeLa cells. (Smith et al. (1999) Proc Natl Acad Sci USA 96: 11134-11139) They reported an increase of kinase activity in the presence of sheared DNA. Using atomic force microscopy, Smith et al. (1999) showed ATM preferentially localizing to ends of DNA double strand gaps, providing some evidence of a protein-DNA interaction. (Smith et al. (1999) Proc Natl Acad Sci USA 96: 11134-11139).

Purification of endogenous ATM by conventional biochemical methods has resulted in extremely low yields of purified protein. Smith and colleagues purified ATM from 50 μg of HeLa cell nuclear extract using a series of chromatography columns (Smith et al. (1999) Proc Natl Acad Sci USA 96: 11134-11139). A double-stranded DNA column was used as the last purification step resulting in a homogenous elution. Atomic force microscopy, used to visualize biological interactions, was used to analyze purified ATM and showed that ATM exists as monomers and tetramers. (Smith et al., (1999) Proc Natl Acad Sci USA 96: 11134-11139)

Chan et al. purified endogenous ATM from human placenta using various biochemical chromatographic steps, resulting in approximately 2 μg of ATM protein from 300 grams of placenta tissue, whereas 500 μg of DNA-protein kinase catalytic subunit (DNA-PKcs) protein was isolated from the same tissue. (Chan et al (2000) Jnl Biol Chem 275: 7803-7810) Rhodes et al. purified FLAG-tagged ATM by transiently transfecting an expression construct in HEK 293T cells and isolating ATM using an anti-FLAG affinity column. (Rhodes et al. (2001) Prot Expression and Purif 22: 462-466) Rhodes et al. were able to purify only 1 μg of ATM protein from a 225 cm² flask that had been seeded with 8×10⁶ uninfected cells and incubated for overnight prior to transfection, and then incubated for another 24 hours after transfection. Thus, the protein recovery reported by Rhodes et al. appeared to be about 1 μg ATM protein from at least 8×10⁶ cells, and relative yield may be even lower if cell division occurred during incubation such that substantially more cells were used for purification. (Rhodes et al. (2001) Prot Expression and Purif 22: 462-466).

Because isolation of the purified ATM protein has been so difficult, assays which use ATM for diagnosing patients have been impractical or even impossible. There exists an unmet need in the art for a method of diagnosing A-T involving an assay which can detect ATM protein levels in a patient. Because of the link between A-T and cancer, particularly breast cancer, there also exists an unmet need for a method of diagnosing cancer susceptibility involving an assay which can detect and/or quantify ATM protein in a patient. Further, there exists an unmet need for an assay which can distinguish between individuals who are homozygous A-T, heterozygous A-T/normal, and homozygous normal. Since the health concerns of individuals in each of those three classes is unique, it would be advantageous to tailor patient counseling, further testing, and medical treatment in light of a patient's A-T genotype.

### SUMMARY OF THE INVENTION

One aspect of the present invention is a method of screening for susceptibility to a disorder in a patient including the steps of: providing a biological sample from the patient; determining the quantity of ataxia-telangiectasia, mutated (ATM) protein in the biological sample; and comparing the quantity of ATM protein in the biological sample to a standard reference quantity of ATM protein, wherein a reduced level of ATM protein in the patient compared to the standard reference level indicates that the patient has an increased susceptibility to the disorder. In preferred embodiments, the disorder is ataxia-telangiectasia, cancer, breast cancer, a neurological disorder, or heart disease. Some preferred embodiments also include the use of an ELISA to quantitate ATM. Preferred biological samples are peripheral blood mononuclear cells or lymphoblastoid cells.

Another aspect of the invention is a method of detecting an ataxia-telangiectasia (A-T) gene mutation in a patient including the steps of: providing a biological sample from the patient; determining the quantity of ATM protein in the biological sample; and comparing the quantity of ATM protein in the biological sample to a standard reference quantity of ATM protein, wherein a reduced level of ATM protein in the patient compared to the standard reference level indicates the presence of an A-T gene mutation in the patient. Such patients can be homozygous A-T, homozygous normal, or heterozygous A-T.

Another aspect of the invention is a method for diagnosing whether a patient has ataxia-telangiectasia, including the steps of: providing a biological sample from the patient; determining the quantity of ATM protein in the biological sample; and comparing the quantity of ATM protein in the biological sample to a standard reference quantity of ATM protein, wherein a reduced level of ATM protein in the patient compared to the standard reference level indicates that the patient has ataxia-telangiectasia.

Another aspect of the invention is a method for producing substantially purified ATM protein including: providing a vaccinia virus vector containing an ATM gene; infecting cells with the vaccinia virus vector; and isolating ATM protein expressed by the cells. Preferably the cells are mammalian cells, more preferably they are HeLa cells. Resins, including FLAG M2 affinity resin can be used to isolate ATM protein.

Another aspect of the invention is a kit for determining the level of ATM protein in a patient, including antibodies that bind to the ATM protein and an assay standard comprising substantially purified ATM protein. Preferably the antibodies are labeled with an enzyme.

Another aspect of the invention is a method of quantitating ATM protein in a biological sample from a patient including: providing a biological sample from the patient, wherein the sample contains ATM protein; providing a standard containing a known amount of ATM protein; and determining the quantity of ATM protein in the biological sample by comparing the biological sample to the standard.

### BRIEF DESCRIPTION OF THE DRAWINGS

FIG. 1 is a line graph showing a dose-response curve for purified ATM protein; the observed spectrophotometric signal is shown as a function of the ATM protein concentration.

FIG. 2 is a graph illustrating the detection of ATM protein in nuclear lysates from cell lines; the ATM protein concentrations for A-T patients are shown in comparison with healthy controls.

FIG. 3 is a three dimensional bar graph showing the stability of ATM protein in whole blood.

FIG. 4 is a three dimensional bar graph showing detection of ATM protein in extracts from PBMC; ATM protein concentration is shown as a function of the quantity of cells used.

### DETAILED DESCRIPTION OF THE PREFERRED EMBODIMENTS

Some embodiments of the invention relate to methods for diagnosing a patient for A-T and/or susceptibility to various conditions. These conditions can include cancer, particularly breast cancer, and heart disease. One embodiment relates to the discovery that persons having an A-T mutation, including A-T heterozygotes, have an increased risk of developing some neurological disorders. Accordingly, susceptibility to these various neurological disorders can also be diagnosed by measuring the level of A-T protein in a patient. Diagnosis is generally performed by detecting levels of ATM protein in a patient at risk for these conditions.

Some embodiments of the invention provide an expression system that produces a high yield of purified functional ATM protein. As used herein, a high yield of functional ATM protein is preferably a yield greater than 2 μg of substantially pure ATM protein per 300 grams fresh weight of host cells or host tissue. This purified functional protein provides a standard level of ATM protein that is used to estimate the quantity of ATM protein in a patient. Of course, a high yield can also mean at least 3 μg of ATM protein, at least 4 μg of ATM protein, at least 5 μg of ATM protein or more per 300 grams fresh weight of host cells or host tissue.

Accordingly, one aspect of the invention is an assay to measure ATM protein levels in a patient. Preferably, cells are taken from a patient and the amount of ATM protein present is determined by an assay. In the assay, the level of ATM protein in the patient is advantageously compared to a known, standard level of ATM protein. In one embodiment, the known, standard level of ATM protein is produced by a vaccinia virus expression system. The results of the assay are used to diagnose whether the patient is “homozygous A-T” (meaning homozygous for the mutated A-T gene), a heterozygous carrier (meaning heterozygous with one mutated A-T gene and one normal A-T gene), or homozygous normal.

Some further embodiments include a method of diagnosing a patient's susceptibility to other conditions, such as cancer, particularly breast cancer, neurological disorders, and heart disease, by measuring the ATM protein levels in a patient suspected of having or developing the condition. Preferably, the assay is a sandwich immunoassay which measures the amount of ATM protein in nuclear cell lysates and cell extracts from the patient's blood by comparing the level of ATM protein in the patient with a known, standard level of ATM. The data derived from the patient's cells are then compared to reference data from cell lines or the cells of other individuals who are either homozygous for the A-T disorder, heterozygous, or homozygous normal. Further, the individuals whose cells are used to prepare reference data may either be healthy or exhibit the condition, such as cancer, that is the subject of the diagnosis. Preferably, the reference data is drawn from a large pool of individuals in which all possible genotypes and phenotypes are represented.

In accordance with the present invention, levels of the ATM protein can be measured in a variety of ways. A preferred type of immunoassay to detect an antibody specific for the ATM protein is an enzyme-linked immunosorbent assay (ELISA) or more generically termed an enzyme immunoassay (EIA). In such assays, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label the reagents useful in the present invention include, but are not limited to, horseradish peroxidase, alkaline phosphatase, glucose oxidase, β-galactosidase, ribonuclease, urease, catalase, malate dehydrogenase, staphylococcal nuclease, asparaginase, DELTA.-5-steroid isomerase, yeast alcohol dehydrogenase, alpha.-glycerophosphate dehydrogenase, triose phosphate isomerase, glucose-6-phosphate dehydrogenase, glucoamylase and acetylcholinesterase. For descriptions of EIA procedures, see Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980; Butler, J. E., In: Structure of Antigens, Vol. 1 (Van Regenmortel, M., CRC Press, Boca Raton, 1992, pp. 209-259; Butler, J. E., In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991)

In some other embodiments, the detectable label may be a radiolabel, and the assay termed a radioimmunoassay (RIA), as is well known in the art. See, for example, Yalow, R. et al., Nature 184:1648 (1959); Work, T. S., et al., Laboratory Techniques and Biochemistry in Molecular Biology, North Holland Publishing Company, NY, 1978, incorporated by reference herein. The radioisotope can be detected by a gamma counter, a scintillation counter or by autoradiography. Isotopes which are particularly useful for the purpose of the present invention are 125 I, 135 I, 35 S, 3H and 14 C.

It is also possible to label the antibody reagents with a fluorophore. When the fluorescently labeled antibody is exposed to light of the proper wave length, its presence can then be detected due to fluorescence of the fluorophore. Among the most commonly used fluorophores are fluorescein isothiocyanate, rhodamine, phycoerythrin, phycocyanin, allophycocyanin, o-phthaldehyde, fluorescamine or fluorescence-emitting metals such as 152 Eu or other lanthanides. These metals are attached to antibodies using metal chelators.

The antibody reagents useful for detecting ATM protein levels can be detectably labeled by coupling to a chemiluminescent compound. The presence of a chemiluminescent-tagged antibody or antigen is then determined by detecting the luminescence that arises during the course of a chemical reaction. Examples of useful chemiluminescent labeling compounds are luminol, isoluminol, theromatic acridinium ester, imidazole, acridinium salt and oxalate ester. Likewise, a bioluminescent compound such as a bioluminescent protein may be used to label antibody reagent. Binding is measured by detecting the luminescence. Useful bioluminescent compounds include luciferin, luciferase and aequorin.

Measuring levels of labeled ATM protein can be carried out by a scintillation counter, for example, if the detectable label is a radioactive gamma emitter, or by a fluorometer, for example, if the label is a fluorophore. In the case of an enzyme label, the ATM protein levels determinations are accomplished by colorimetry to measure the colored product produced by conversion of a chromogenic substrate by the enzyme. Detection may also be accomplished by visual comparison of the colored product of the enzymatic reaction in comparison with appropriate standards or controls.

The immunoassay may be a “two-site” or “sandwich” assay. The fluid containing the antibody being assayed is allowed to contact a solid support. After addition of the biological sample containing the ATM protein, a quantity of detectably labeled soluble antibody is added to permit detection and/or quantitation of the ternary complex formed between solid-phase antibody, ATM protein, and labeled antibody. Sandwich assays are described by Wide, Radioimmune Assay Method, Kirkham et al., Eds., E. & S. Livingstone, Edinburgh, 1970, pp 199-206.

Alternatives to the RIA and EIA are various types of agglutination assays, both direct and indirect, which are well known in the art. In these assays, the agglutination of particles containing the ATM protein (either naturally or by chemical coupling) indicates the presence or absence of the corresponding antibody. Any of a variety of particles, including latex, charcoal, kaolinite, or bentonite, as well as microbial cells or red blood cells, may be used as agglutinable carriers (Mochida, U.S. Pat. No. 4,308,026; Gupta et al., J. Immunol. Meth. 80:177-187 (1985); Castelan et al., J. Clin. Pathol. 21:638 (1968); Singer et al., Amer. J. Med.(December 1956, 888; Molinaro, U.S. Pat. No. 4,130,634). Traditional particle agglutination or hemagglutination assays are generally faster, but much less sensitive than RIA or EIA. However, agglutination assays have advantages under field conditions and in less developed countries.

In some other embodiments, a capture enzyme-linked immunosorbent assay (ELISA) method includes the use of two (monoclonal or polyclonal) antibodies to the same antigen with two different epitopes, one of which is conjugated with biotin. Biological samples containing ATM protein can be reacted with the first antibody and washed with a buffer solution. The antibody linked to ATM can then be reacted with the second antibody which is conjugated with biotin-N-hydroxy succinamide and then washed to remove the excess antibody. The antibody-biotin-antibody linked ATM protein can then be cross-linked with avidin-peroxidase and washed to remove the excess antibody. Finally, a substrate can be reacted with the avidin-peroxidase-crosslinked-antibody-biotin (B)-antibody (A) linked antigen, the color product of which upon development is measured by O.D. with an ELISA reader. It will be appreciated that a variety of antibodies and techniques known in the art are suitable for this procedure.

Embodiments of the invention can also be directed to a kit or reagent system useful for practicing the methods described herein. Such a kit will generally contain a reagent combination comprising the essential elements required to conduct an assay according to the disclosed methods. The reagent system can be presented in a commercially packaged form, as a composition or admixture (where the compatibility of the reagents allow), in a test device configuration, or more typically as a test kit. A test kit is typically a packaged combination of one or more containers, devices, or the like holding the necessary reagents, and usually including written instructions for the performance of assays. The kit may include containers to hold the materials during storage, use or both. The kit may include any configurations and compositions for performing the various assay formats described herein.

For example, a kit for determining the presence of the ATM protein in a biological sample from a patient may contain an immobilizable or immobilized “capture” antibody which reacts with one epitope of the ATM protein, and a detectably labeled second (“detection”) antibody which reacts with a different epitope of the ATM protein than that recognized by the (capture) antibody. Any conventional tag or detectable label may be part of the kit, such as a radioisotope, an enzyme, a chromophore or a fluorophore. The kit may also contain a reagent capable of precipitating immune complexes.

A kit according to the present invention can additionally include ancillary chemicals such as the buffers and components of the solution in which binding of antigen and antibody takes place.

ATM Expression by Vaccinia Virus

Construction of Recombinant ATM-Expressing Vaccinia Virus

Since cellular ATM levels are inherently low, a recombinant vaccinia virus system that expresses ATM was used to produce a high yield of ATM protein. Full-length human ATM cDNA (GenBank Accession No. U33841) was inserted into the pSC65 vaccinia vector (Chakrabarti et al (1997) Biotechniques 23: 1094-1097) containing a synthetic “early/late promoter” having both early and late gene promoters such that an insert is expressed throughout the virus life cycle, and also containing the tk (thymidine kinase) gene. Insertion of full-length ATM into pSC65 produced the pSCAT expression vector, which was transfected into CV-1 tk− cells from a monkey kidney cell line that were simultaneously transfected with the WR strain of vaccinia virus (WR strain: ATCC VR 1354), and ATM-encoding polynucleotide was incorporated into the viral genome at the tk gene locus, preferably by homologous recombination.

Double selection was performed to isolate a single population of recombinant virus. For double selection, ATM was inserted into the viral genome by homologous recombination at the tk locus. The first selection involved a negative tk selection to determine if homologous recombination took place between the vaccinia vector and the viral genome, as a cell expressing thymidine kinase gene will be killed in the presence of bromodeoxyuridine (BrdU). The CV-1 cell line lacks thymidine kinase (tk−) and the vector has the tk gene. In this embodiment, ATM is inserted into the tk gene, making tk nonfunctional. After introduction of ATM into the viral genome, recombinant viruses with successful homologous recombination are identified by having a tk− phenotype. A second selection step uses color, preferably lacZ, to select for transfected tk− cells.

Recombinant ATM-expressing vaccinia virus was then recovered from infected cells and used to infect other host HeLa cells (ATCC Accession No CCL2.2). The host cells were then used for maintaining or propagating stocks of infected cells for future use and for preparation of amplification stock for purification or measurements of ATM protein.

Purification of ATM

Substantially pure whole ATM will yield a single major band of about 370 kDa on a denaturing polyacrylamide gel. The purity of compositions containing ATM can also be determined by amino-terminal amino acid sequence analysis.

Recombinant ATM may be purified by any suitable method, including but not limited to chromatography, precipitation, electrophoresis, and if desired, combinations of various methods. Chromatographic techniques suitable for ATM purification include ion exchange chromatography, affinity chromatography, size-exclusion, chromatography, using liquid chromatographic systems such as HPLC or gas chromatographic systems. ATM purification may be isolated by precipitation, for example immunoprecipitation using anti-ATM antibody, using calcium, or using an antibody against a “tag” group attached to ATM. Electrophoretic methods suitable for ATM purification include but are not limited to isoelectric focusing, polyacrylamide gel electrophoresis under nondenaturing or denaturing conditions, agarose gel electrophoresis, iontophoresis, or other electrophoretic methods of protein separation.

Recombinant ATM can be made as a fusion protein having a FLAG tag at the N-terminal end of the protein. Alternatively, recombinant ATM can be made as a fusion protein having both FLAG and hexahistidine (HIS) tags located at the N-terminal end of the protein.

In one experiment, HeLa cells were infected with ATM vaccinia virus for 32 hours and lysed to release ATM. Cytoplasmic extracts from cells infected with ATM-expressing virus were incubated in small batches with FLAG M2 affinity resin (Sigma), under suitable conditions to allow ATM to bind to the resin. FLAG-tagged ATM was eluted from the affinity resin by peptide competition using 1 mg/ml FLAG peptide (Sigma). Typical yields of substantially purified ATM were between 0.3-0.5 μg/μl of eluate from FLAG M2 resin. After elution, eluate was optionally concentrated using Microcon YM-100 centrifugal filter (Amicon). Western blot analysis using anti-ATM antibodies or anti-FLAG antibodies confirmed the presence of ATM in the eluate. Silver-stained protein showed that most of the protein present in the concentrated eluate was full-length ATM, although traces of smaller protein fragments at much lower concentrations were also detected.

In accordance with some embodiments of the present invention, high yields of ATM protein are produced using an expression system as disclosed herein, where ATM protein is preferably recovered in substantially purified form. Yields are greater than 2 μg substantially purified ATM from 300 grams of tissue, or greater than 1 μg substantially purified ATM following several days of growth cycles starting from 8×106 cells. Preferably, yields of at least 2 μg, preferably 5 μg, even more preferably 10 μg, and even more preferably 20 μg or 25 μg or 30 μg or more of substantially purified ATM is recovered from 8×10⁶ infected cells. In some embodiments, approximately 500 μl FLAG M2 resin eluate is collected from about 25×10⁶ infected HeLa cells at a concentration of about 0.4-0.5 mg protein/ml of eluate, giving a total yield of about 200-250 μg substantially pure ATM. In other embodiments, approximately 100 μl of FLAG M2 resin eluate is collected from 8×10⁶ infected cells at a concentration of about 0.2-0.3 mg/ml of eluate, giving a total yield of about 45 μg of substantially pure ATM. One of skill in the art can optimize yield according to the infected host cells or tissue used, the equipment and reagents available, purification methods used, and degree of purity desired.

The present disclosure enables one of skill in the art to adapt the ATM expression system in order to purify recombinant ATM protein by any desired method. For example, expression vectors can be constructed to attach a glutathione-S-transferase (GST) tag to the ATM protein, and GST-tagged ATM can be affinity-purified. Further, one of skill in the art can carry out additional manipulations to recover ATM in the desired form. For example, a composition of substantially purified affinity-tagged ATM can be treated to remove the affinity tags, e.g., GST tags may be removed by proteolytic cleavage with enterokinase or thrombin. For ATM proteins having multiple tags, tags may be selectively removed if desired, e.g., a GST-and-FLAG-tagged ATM may be treated with thrombin to remove the GST tag, while the FLAG tag remains attached. Alternately, self-cleaving tags such as the intein system may be used to substantially purify ATM protein and then remove the affinity tag used for purification.

The present disclosure describes production and purification of functional ATM, preferably by over-expression of ATM, preferably using vaccinia virus as the expression system. Use of vaccinia virus permits expression in mammalian hosts, which can be advantageous when compared to the baculovirus expression system. The inability of insect cells to mass produce the large protein may be due to amino acid differences or lethal effects to the host due to large quantities of expressed protein. As exemplified by the present disclosure, use of mammalian hosts diminished the problem of rare codons. However, one of skill in the art could practice the vaccinia viral expression method disclosed herein using non-mammalian cells including insect cells, possibly by modifying codon usage in the ATM-encoding polynucleotide.

Cytoplasmic transcription is an especially advantageous property of the vaccinia virus with respect to some aspects of the present invention. Transcription of viral RNA outside the host cell nucleus avoids the problem of incorrect RNA splicing. Given the large size of the ATM cDNA, this may be a problem in non-mammalian cells.

Diagnosing Conditions Based on ATM Protein Levels

As discussed above, some embodiments of the invention include a diagnostic assay that measures the amount of ATM protein in cells extracted from a patient by comparing the patient's ATM levels with a known, standard level of ATM. The amount of ATM protein present in patient populations has been found to be directly correlated with whether or not a patient had A-T. For example, it has been estimated that 80% of all A-T gene mutations lead to a truncated ATM protein. In addition, other A-T gene mutations lead to an ATM protein that is unstable in the body and therefore quickly degrades.

Moreover, assays for ATM can be used to determine whether a patient has an A-T gene mutation. Specifically, an assay can be used to characterize whether the patient has an A-T gene mutation that results in an unstable protein, which is presumed to be degraded in the body. For this reason, patients having a mutated ATM gene typically have lower cellular levels of ATM protein. The assay can therefore be performed by comparing the ATM protein levels taken from the patient with standard reference data on the amounts of ATM protein present in normal individuals, heterozygotes for an A-T gene mutation, and homozygotes for an A-T gene mutation.

Further, because A-T gene mutations have been linked to susceptibility for other conditions, including for example, cancer, particularly breast cancer, neurological disorders, and heart disease, measuring the amount of ATM protein in a patient's cells can be predictive for a patient's susceptibility for such a condition. Preferably the reference data is compiled from a large pool of individuals representing all possible A-T genotypes and phenotypes. For example, where reference data indicates that individuals having a lower-than-normal concentration of ATM protein show an increased incidence of breast cancer, this would suggest an increased susceptibility to breast cancer for a patient exhibiting a similar, lower-than-normal concentration of ATM protein.

Definitions

The ATM protein and other materials can advantageously be in isolated form. As used herein, the term “isolated” denotes that the material has been removed from its original environment. For example, a naturally-occurring polynucleotide or polypeptide present in a living animal is not isolated, but the same polynucleotide or polypeptide, separated from some or all of the coexisting materials in the natural system, is isolated.

It is also advantageous that the sequences and other materials comprising the invention be in purified form. The term “purified” does not require absolute purity; rather, it is intended as a relative definition. For purposes of clarity, the term “substantially purified” or “substantially pure” is used herein to indicate that absolute purity is not required. A “substantially purified” or “substantially pure” substance therefore can be a mixture in which the substance is the merely the predominant species; one or more impurities may be present. Purification of starting material or natural material means that the concentration of the substantially purified material is at least about 2, 5, 10, 100 or 1000 times its original concentration (for example), advantageously 0.01% by weight, preferably at least about 0.1% by weight. Purified preparations of about 0.5%, 1%, 5%, 10%, 20%, and 40% by weight are also contemplated.

### EXAMPLES

### Example 1

### Production of ATM Protein

Cell Culture and Irradiation

CV-1 tk− cells were maintained in DME (Hyclone) supplemented with 10% fetal calf serum (Hyclone). The cells were grown in a humidifying incubator at 37° C. with 5% CO₂. HeLa cells were maintained in DMEM (Cellgro) supplemented with 10% fetal bovine serum (Hyclone) and 1% penicillin/streptomycin/glutamine (Gibco BRL) and human lymphoblastoid cells, L3, were maintained in RPMI (Cellgro) supplemented with 10% fetal bovine serum and 1% penicillin/streptomycin/glutamine. The cells were grown in a humidifying incubator at 37° C. with 5% CO₂. Cells treated with irradiation were exposed to 2 Gy gamma radiation. Cells infected with vaccinia virus were returned to 37° C. after infection until lysis.

Construction of pSCAT Vector

pFT-YZ5, a baculovirus construct containing the full-length ATM cDNA, was generously donated by Yosef Shiloh. Directly flanking the 5′ end of the ATM coding sequence are sequences coding for the FLAG epitope and hexahistidine tags. Liberation of the entire ATM coding sequence, including the FLAG and HIS tags, was performed by a SalI and KpnI (New England Biolabs) double digestion, resulting in a 5′ piece of ATM of 4 kb and a 3′ fragment of 5.7 kb. The 5′ ATM fragment was inserted into the vaccinia vector pSC65 at the SalI and KpnI sites, producing pSC-5ATM. The 3′ ATM piece was ligated into pSC-5ATM at KpnI and checked with restriction enzymes for insertion in the correction orientation. DNA sequencing was performed to ensure the integrity of all ligation sites. The final construct, pSCAT, is approximately 16.6 kb. All plasmids were grown in MAX DH5 alpha cells (Gibco BRL) at 30° C.

Construction of Recombinant ATM Vaccinia Virus

CV-1 tk− cells were infected with WR strain of vaccinia virus at an MOI=0.1 pfu/cell for 2 hours followed by transfection of pSCAT using lipofectin (Gibco BRL). After 48 hours, cells were collected, resuspended in 1 ml OptiMEM (Gibco BRL), sonicated, and plated at 10⁻² to 10⁻⁴ dilutions on tk− cells plated on 6-well plates to undergo selection for recombinant virus. A first overlay containing Basal Medium Eagle (Gibco BRL), L-glutamine, 0.05 mg/ml 5-bromo-2-deoxyuridine, 5% fetal bovine serum, and 1% low melting point agarose (BRL), was placed 2 hours after infection. The second overlay, containing 5 μg/ml neutral red, 0.002% x-galactose (Fisher), Basal Medium Eagle, and 1% LMP agarose, was placed 48 hours after infection. Within 36 hours, blue plaques were picked with a Pasteur pipette and placed into 500 μl OptiMEM and sonicated. Repeated plaque selection was performed until a purified virus was obtained.

Immunoblot Analysis of Expression

Lysates were prepared using lysis buffer containing 50 mM Tris HCl pH 7.4, 150 mM NaCl, 2 mM EDTA, 0.2% Triton X-100, 0.3% NP-40, 5 μg aprotinin (Sigma), 5 μg leupeptin (Calbiochem) and 1 mM PMSF (Sigma), incubated on ice and cleared by centrifugation. Cytoplasmic extract containing virally expressed ATM was prepared and run on a 5% denaturing polyacrylamide gel. To observe p53 phosphorylation, sonication was used to prepare nuclear extracts followed by electrophoresis on a 6 or 7% denaturing gel. SDS-PAGE gels were transferred for 2 hours at 100V, incubated with anti-ATM (Novus), anti-FLAG M2 (Sigma), or anti-phospho-p53 serine 15 (Cell Signaling) antibodies. Protein were visualized using enhanced chemiluminesence (Amersham).

Immunoprecipation and in Vitro Kinase Assay

Lysates were prepared as previously described and brought to a final volume of 800 μl. 5 μg of FLAG M2 antibody (Sigma) was used to immunoprecipitate the recombinant ATM and captured with Protein G Plus beads (Santa Cruz Biotechnology). In vitro kinase assay was performed using 50 mM HEPES pH 7.5, 150 mM NaCl, 10 mM MnCl₂, 10 mM MgCl₂, 1 mM DTT plus protease inhibitors, and 2 μg GST-p53 (Santa Cruz Biotechnology) or PHAS-1 (Stratagene), in the presence or absence of 10 μg of sheared Salmon sperm DNA (Stratagene), and pre-incubated for 3 minutes on ice. Upon addition of 20 μCi γ-³³P-ATP (3000 Ci/mmol, Perkin Elmer) and 6.7 μM ATP, the kinase reaction was incubated at 30° for 15 minutes and stopped with SDS sample buffer. The reaction was run on a 7% SDS-PAGE gel, dried, and exposed to film. For DNase treated reactions, 10U of DNase (Gibco BRL) was added to the corresponding samples followed by a 37° C. incubation of all samples for 15 minutes. Wortmannin (Sigma), at a final concentration of 5 mM, was incubated with ATM prior to ATP addition for 30 minutes at room temperature.

### Example 2

### Purification of Recombinant ATM

FLAG M2 affinity resin (Sigma) was washed several times with lysis buffer. Approximately 25×10⁶ HeLa cells were infected with recombinant vaccinia virus at MOI=5 pfu/cell for 32 hours. Cells were lysed with 2 ml lysis buffer, incubated for 15 minutes on ice, and cleared by centrifugation. Cytoplasmic protein was incubated with 400 μl packed FLAG M2 affinity resin for 2 hours on rocker. Resin was collected by centrifugation for 2 minutes at 8000 rpm and washed with lysis buffer. 1 mg/ml FLAG peptide (Sigma) eluted ATM by peptide competition when incubated on rocker for 1 hr. Eluates were concentrated using a Microcon YM-100 centrifugal filter (Amicon). Final concentration of substantially purified ATM was typically between 0.3 to 0.5 mg/ml in the eluate. All purification steps were performed at 4° C.

### Example 3

### Activity of Purified ATM

Activity of substantially purified ATM protein was measured using an in vitro kinase assay. The assay contained ATM from Example 2 in the presence of 50 mM HEPES pH 7.5, 150 mM NaCl, 10 mM MnCl₂, 10 mM MgCl₂, 1 mM DTT plus protease inhibitors, and 2 μg GST-p53 (Santa Cruz Biotechnology) or PHAS-1 (Stratagene), in the presence or absence of 5 μg of sheared Salmon sperm DNA (Stratagene), and was pre-incubated for 3 minutes on ice. Upon addition of 20 μCi γ-³³P-ATP (3000 Ci/mmol, Perkin Elmer) and 6.7 μM ATP, the kinase reaction was incubated at 30° for 15 minutes and stopped with SDS sample buffer. The reaction was run on a 7% SDS-PAGE gel, dried, and exposed to film. For DNase treated reactions, 10 units of DNase (Gibco BRL) was added to the corresponding samples followed by a 37° C. incubation of all samples for 15 minutes. Wortmannin (Sigma), at a final concentration of 5 mM, was incubated with ATM prior to ATP addition for 30 minutes at room temperature.

### Example 4

### Detecting ATM Protein in a Patient

The following is a sandwich immunoassay which was performed to measure ATM protein in nuclear cell lysates and cell extracts from cell lines and peripheral blood mononuclear cells taken from normal individuals and A-T patients.

Wells of a 96-well flat bottom plate were incubated with two purified commercially available anti-ATM mouse monoclonal antibodies (ATM-2C1, GeneTex, Inc, San Antonio, Tex.; Ab-8, NeoMarkers, Fremont, Calif.) at 5 μg/ml in phosphate-buffered saline, pH 7.4 for 6 hours. After extensive washing and blocking of the wells for one hour with a solution of 3% bovine serum albumin in phosphate buffered saline containing 0.1% Tween-20, standard concentrations of purified ATM protein and nuclear cell lysates or cell extracts (see below) were added to the wells for an overnight incubation at room temperature.

After extensive washing, a rabbit anti-ATM antiserum at a 400× dilution was added to each of the wells and incubated for 3 hours at room temperature. The antiserum came from Novus Biologicals (NB 100-104, Littleton, Colo.). After washing and blocking, an HRP-conjugated goat anti-rabbit IgG antiserum at a 1:6000 dilution was added to each well and incubated for 3 hours at room temperature. The conjugate came from Jackson ImmunoResearch Laboratories (211-035-109, West Grove, Pa.).

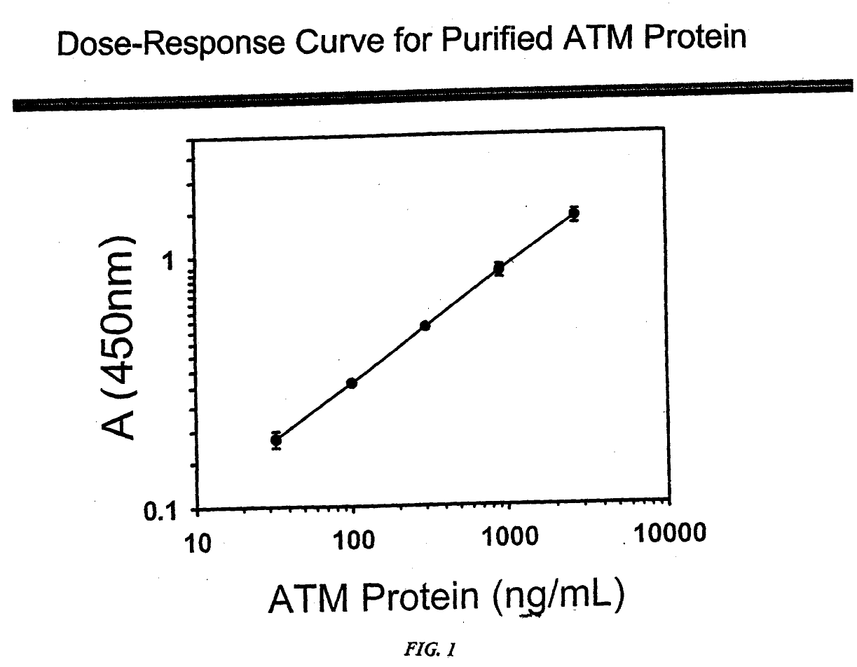

The plate was then washed and Pierce ImmunoPure tetramethylbenzidine (TMB) substrate (34021, Rockford, Ill.) was added to each well. After color development, the reaction was stopped with 1M sulfuric acid and the color intensity was measured spectrophometrically at 450 nm. A concentration curve was generated based on signals generated for the purified ATM protein and concentrations of unknown samples were read off the standard curve. The standard curve is shown in FIG. 1.

Nuclear lysates were prepared from lymphoblastoid cell lines (EBV-infected) derived from A-T patients and healthy donors using commercially available extraction reagents and procedures (78833, Pierce, Rockford, Ill.). Whole cell extracts were generated from peripheral blood mononuclear cells and lymphoblastoid cell lines by subjecting known numbers of resuspended cells to ultrasonic energy at a 20 kHz frequency generated by a Fisher Sonic Dismembrator (model 550). The disrupted cell product was then added directly to wells of the microtiter plate without any additional manipulations.

For purified ATM protein, the immunoassay was linear from 33 to 2700 ng/mL (as shown in FIG. 1). Intra-assay precision at a mean target value of 253 (n=10) and 806 (n=8) was 7.4% and 6.8% while total imprecision at a mean value of 122 (n=12) and 456 (n=12) were 21.2% and 15.1%, respectively; these results are illustrated in Table 1.

TABLE 1
Precision Studies

| | | mean | | |
|---|---|---|---|---|
| | n | (ng/mL) | SD | % CV |
| Intra-assay | 10 | 253 | 19 | 7.4 |
| | 8 | 806 | 54 | 6.8 |
| Inter-assay | 12 | 122 | 26 | 21.2 |
| | 12 | 456 | 69 | 15.1 |

Nuclear cell lysates from lymphoblastoid cell lines derived from A-T patients were tested and 10 of the 12 had undetectable levels of ATM protein (<33 ng/mL). One patient had a value of 34 and the other a value of 84 ng/mL.

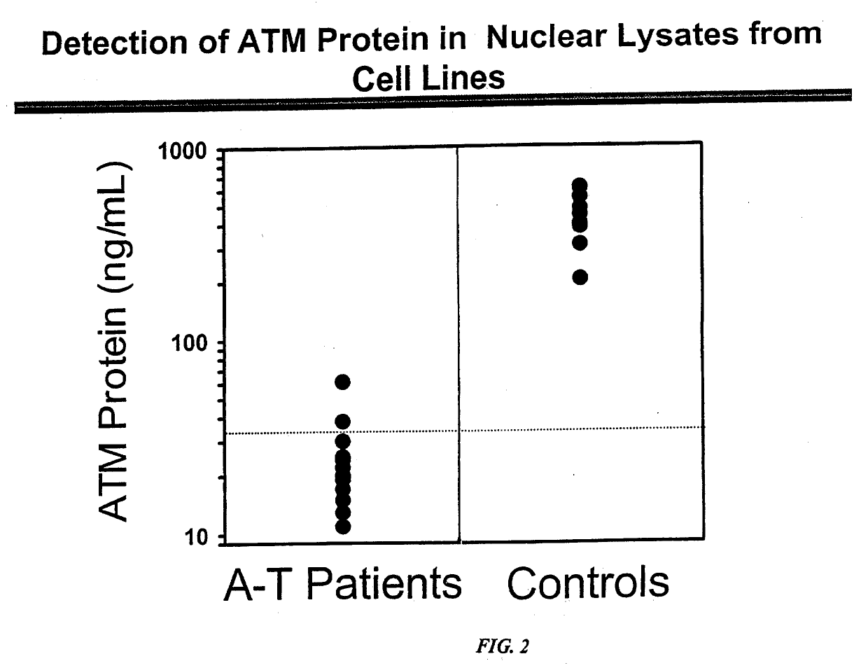

Nuclear cell lysates (40 micrograms of total protein) from cell lines derived from healthy controls had ATM protein concentrations ranging from 204 to 610 ng/mL. The measured protein concentrations for the A-T patients are shown against those of the healthy controls in FIG. 2.

Next, peripheral blood mononuclear cells (PBMCs) from nine healthy controls were evaluated for levels of ATM protein. Using 40 micrograms of PBMC cell lysate protein in the immunoassay, it was found that ATM concentrations ranged from 48 to 943 ng/mL.

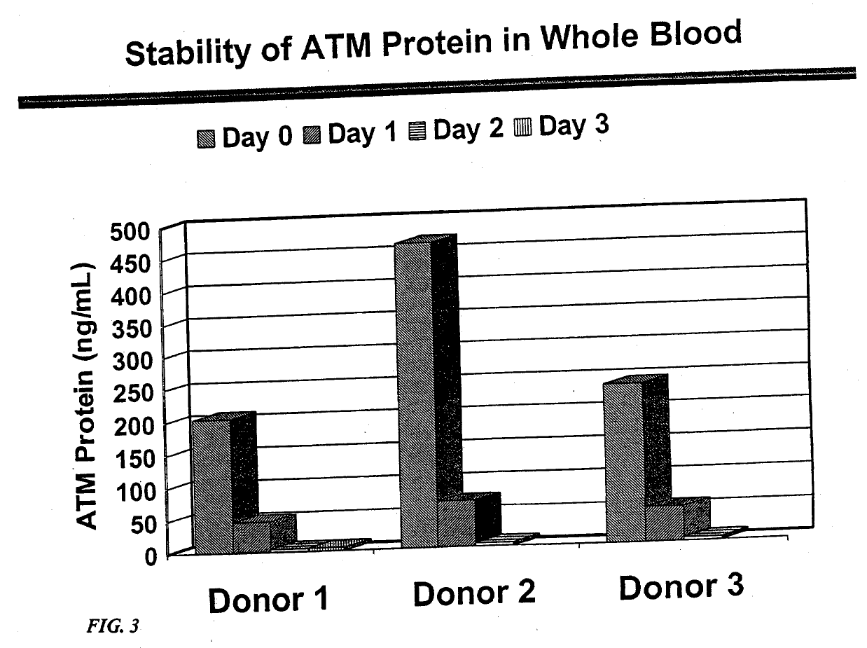

It has been observed that ATM protein is unstable in whole blood. FIG. 3 shows that ATM protein levels drop substantially in the first day and can fall below detectable levels within a few days. It is therefore preferable that ATM proteins be isolated and measured shortly after extracting the cells from a patient to obtain the most reliable data. It has been discovered, however, that ATM protein levels can be made more stable if peripheral blood mononuclear cells (PBMCs) are isolated from fresh blood and stored at −70° C. Table 2 compares the amount of ATM in two such samples stored at −5° C. and −70° C. As shown here, ATM levels in a sample can remain relatively stable for 6 weeks when stored at −70° C.

TABLE 2
Comparison of ATM Samples Stored at Different Temperatures

| Week | −5° C. (3 readings) | −70° C. (3 readings) |
|---|---|---|
| 0 | 444 ± 22 | 444 ± 22 |
| 1 | 126 ± 8 | 458 ± 11 |
| 2 | 115 ± 14 | 512 ± 63 |
| 3 | 66 ± 17 | 424 ± 34 |
| 4 | 50 ± 10 | 439 ± 79 |
| 6 | 32 ± 2 | 458 ± 69 |

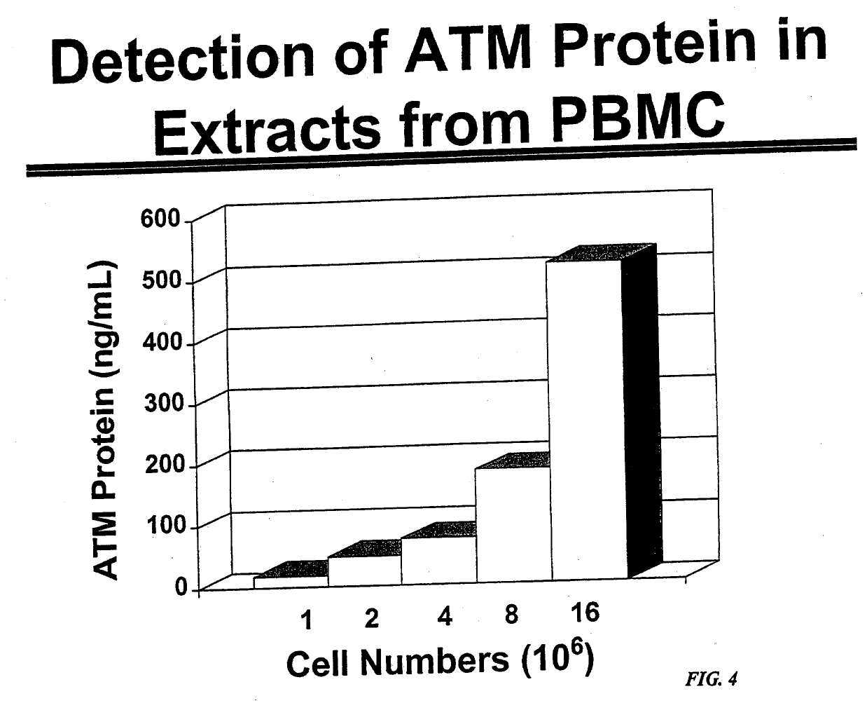

Using a Sonic Dismembrator (Fisher Scientific, Pittsburgh, Pa.), cell extracts from 1×10⁶, 2×10⁶, 4×10⁶, 8×10⁶, and 16×10⁶ cells were found to contain 18, 48, 74, 183 and 516 ng/mL of ATM protein, respectively. These results appear in FIG. 4.

### Example 5

### Developing a Statistical Toll for Testing Susceptibility to Breast Cancer

Nuclear cell lysates from lymphoblastoid cell lines derived from patients at risk for breast cancer are tested to measure the amount of ATM protein present. Additionally, ATM protein from nuclear cell lysates of lymphoblastoid cell lines derived from individuals who are not at risk for breast cancer are used as controls. ATM protein levels are measured as described above.

It has been observed that the ATM protein concentration in healthy individuals is higher on average than ATM protein concentration in patients that develop breast cancer. A statistical range of ATM protein concentration can be determined for healthy individuals. A second statistical range of ATM protein concentration can also be determined for individuals diagnosed with breast cancer.

It is recognized that there are three genotypes related to the A-T gene: homozygous A-T, heterozygous, and homozygous normal. These genotypes correlate to individuals having either of two manifestations: healthy individuals and those with a higher risk of developing breast cancer. The breast cancer manifestation can be further defined by degree, however. Breast cancer and its severity is statistically correlated to the different genotypes. Although any genotype can produce healthy individuals as well as those with breast cancer, it is observed that homozygous normal individuals have the lowest incidence of breast cancer in comparison to individuals heterozygous or homozygous for the A-T gene. Additional statistical ranges can be established to correlate the three different genotypes with rates of occurrence and/or severity of breast cancer.

Some or all of the statistical ranges are combined to create a tool for determining the likelihood that a patient having a given ATM protein concentration will develop breast cancer. This tool is embodied in a chart, a book, a mathematical formula or algorithm, a computer program, or other appropriate medium. Such a tool can also take into consideration other data or patient information that is believed to be relevant to breast cancer susceptibility.

### Example 6

### Screening a Patient for Susceptibility to Breast Cancer

A new patient suspected of being susceptible to breast cancer is identified. Nuclear cell lysates derived from the new patient's cells are tested to measure the amount of ATM protein present. This level is compared with known levels of ATM protein from homozygous normal controls. The diagnostic tool described above is then used to determine whether the patient is at an increased risk of developing breast cancer based on the results of the ATM protein assay.

This information is combined with other factors known or suspected to be related to an individual's susceptibility to breast cancer (including family history, age, diet, status as a smoker, ethnicity, geographic and/or environmental factors, etc.) to generate an overall prediction of the patient's susceptibility to breast cancer. This overall prediction information is then used for patient counseling, further testing, and/or medical treatment as deemed necessary. These steps allow the patient to have more information about her particularized risk for breast cancer and allow her to take actions which can lead to a healthier and longer life.

This procedure is performed on individuals believed to be at increased risk for breast cancer. This increased risk can be based on family history of breast cancer, family history of A-T or A-T carriers, or on other factors known or suspected to be related to breast cancer. Alternatively, the procedure can be performed on any individual to assist in calculating the individual's risk of developing breast cancer, or of having children who may develop breast cancer.

Further, the invention can be used to assess risks of developing other conditions that are found to be related to ATM protein levels. These other conditions can include various forms of cancer, neurological disorders, and heart disease, particularly ischemic heart disease. Any other condition that is actually or theoretically correlated to the A-T gene and/or the ATM protein may also be considered.

Although the invention has been described with reference to embodiments and examples, it should be understood that various modifications can be made without departing from the spirit of the invention. Accordingly, the invention is limited only by the following claims.

What is claimed is:

1. A method of screening for susceptibility to a disorder in a patient comprising:
providing a biological sample from the patient;
isolating a quantity of ataxia-telangiectasia, mutated (ATM) protein in said biological sample; and
comparing the quantity of ATM protein in the biological sample to a standard reference quantity of ATM protein, wherein a reduced level of ATM protein in the biological sample compared to the standard reference quantity indicates that the patient has an increased susceptibility to the disorder.

2. The method of claim 1 wherein the disorder is ataxia-telangiectasia.

3. The method of claim 1 wherein the disorder is cancer.

4. The method of claim 1 wherein the disorder is breast cancer.

5. The method of claim 1 wherein the disorder is a neurological disorder.

6. The method of claim 1 wherein the disorder is heart disease.

7. The method of claim 1 wherein comparing the quantity of ATM protein comprises an ELISA.

8. The method of claim 1, wherein the biological sample comprises peripheral blood mononuclear cells.

9. The method of claim 1, wherein the biological sample comprises lymphoblastoid cells.

10. A method of detecting an ataxia-telangiectasia (A-T) gene mutation in a patient comprising:
providing a biological sample from the patient;
isolating a quantity of ATM protein in said biological sample; and
comparing the quantity of ATM protein in the biological sample to a standard reference quantity of ATM protein, wherein a reduced level of ATM protein in the biological sample compared to the standard reference quantity indicates the presence of an A-T gene mutation in the patient.

11. The method of claim 10, wherein the patient is homozygous for the A-T gene mutation.

12. The method of claim 10, wherein the patient is homozygous normal with respect to the A-T gene mutation.

13. The method of claim 10, wherein the patient is heterozygous for the A-T gene mutation.

14. The method of claim 10, wherein comparing the quantity of ATM comprises an ELISA.

15. The method of claim 10, wherein the biological sample comprises peripheral blood mononuclear cells.

16. The method of claim 10, wherein the biological sample comprises lymphoblastoid cells.

17. A method for diagnosing whether a patient has ataxia-telangiectasia, comprising:
providing a biological sample from the patient;
isolating a quantity of ATM protein in said biological sample; and
comparing the quantity of ATM protein in the biological sample to a standard reference quantity of ATM protein, wherein a reduced level of ATM protein in the biological sample compared to the standard reference quantity indicates that the patient has ataxia-telangiectasia.

18. The method of claim 17, wherein comparing the quantity of ATM protein comprises an ELISA.

19. The method of claim 17, wherein the biological sample comprises peripheral blood mononuclear cells.

20. The method of claim 17, wherein the biological sample comprises lymphoblastoid cells.

21. A method for producing substantially purified ATM protein comprising:
providing a vaccinia virus vector comprising an ATM gene;
infecting cells with said vaccinia virus vector; and
isolating ATM protein expressed by the cells.

22. The method of claim 21, wherein the cells are mammalian cells.

23. The method of claim 21, wherein the cells are HeLa cells.

24. The method of claim 21, wherein isolating ATM protein comprises using a resin capable of binding to said ATM protein.

25. The method of claim 24, wherein the resin is a FLAG M2 affinity resin.

26. The method of claim 21, wherein the A-T gene is operably linked to a promoter.

27. A kit for determining the level of ATM protein in a patient, comprising:
antibodies that bind to the ATM protein; and
an assay standard comprising substantially purified ATM protein.

28. The kit of claim 27, wherein said antibodies are labeled.

29. The kit of claim 28, wherein the antibodies are labeled with an enzyme.

30. A method of quantitating ATM protein in a biological sample from a patient comprising:
providing a biological sample from the patient, wherein the sample comprises ATM protein;
providing a standard comprising a known amount of ATM protein; and
determining the quantity of ATM protein in the biological sample by comparing the biological sample to the standard.

31. The method of claim 30 wherein determining the quantity of ATM protein in the biological sample comprises attaching a label to ATM protein.

32. The method of claim 30 wherein determining the quantity of ATM in the biological sample comprises spectrophotometric, fluorometric, or visual analysis.

33. The method of claim 30 wherein determining the quantity of ATM in the biological sample comprises measuring color intensity.
